(19) United States
(12) Patent Application Publication
(10) Pub. No.: US 2026/0092910 A1
(43) Pub. Date: Apr. 2, 2026

(54) WATER DISPENSING DEVICE

(71) Applicant: LG ELECTRONICS INC., Seoul (KR)

(72) Inventors: Ilna SON, Seoul (KR); Chulwoo PARK, Seoul (KR); Yumin KIM, Seoul (KR); Sangjoon LEE, Seoul (KR)

(21) Appl. No.: 19/113,542

(22) PCT Filed: Sep. 7, 2023
(86) PCT No.: PCT/KR2023/013398

§ 371 Date: Mar. 20, 2025

(30) Foreign Application Priority Data
Sep. 22, 2022 (KR) 10-2022-0120224

(51) Int. Cl. G01N 33/18; E03C 1/044; G01N 21/49

(52) U.S. Cl. CPC G01N 33/18; E03C 1/044; G01N 21/49; E03C 2201/40

(57) ABSTRACT

A water dispensing device, according to one embodiment of the present disclosure, includes: a water supply channel through which raw water supplied from a water supply source flows; a filter which generates purified water by filtering the raw water supplied through the water supply channel; a purified water channel through which the purified water passed through the filter flows; and a water quality measuring unit which is connected to the purified water channel to measure a water quality of the purified water, wherein the water quality measuring unit includes a turbidity sensor, and the turbidity sensor collects N pieces of turbidity data during a first sensing time, outputs a sensing result on the basis of the N pieces of turbidity data if there is no abnormality in the collected N pieces of turbidity data, and re-collects the turbidity data to determine whether there is an abnormality if there is an abnormality in the collected N pieces of turbidity data.

### TECHNICAL FIELD

The present disclosure relates to a water dispensing device and an operating method thereof, and more particularly, to a water dispensing device including a sensor capable of determining a water quality, and an operating method thereof.

### BACKGROUND ART

A water dispensing device supplies water and may take out a desired amount of water at a desired temperature according to the manipulation of a user. The water dispensing device as such may be applied to various fields, but may be applied representatively to a refrigerator and a water purifier. In particular, the water dispensing device provided in the refrigerator and the water purifier is configured to have a function that enables supplying of a preset amount of water according to the manipulation of a user. Recently, for these water dispensing devices, the water dispensing devices that may supply not only purified water but also cold and hot water have been developed.

For example, a water purifier is connected to a water supply source such as a tap to receive raw water, uses a filter to remove floating matters or harmful substances contained in the raw water, and is configured to purify and take out a desired amount of water according to the manipulation of a user. A variety of water purifier products are being released that may purify water as well as heat or cool the purified water to supply cold or hot water. In addition, recently, water purifiers that are small in size and may be installed in various installation environments are being developed.

In a case where the water dispensing device is used for a long time, microorganisms or the like may be propagated in or contaminate pipes, valves, or water outlets. In addition, due to the elapse of the replacement cycle of a filter, floating matters or harmful substances contained in raw water may not be removed. Accordingly, it is important for the water dispensing device to accurately measure water quality, perform hygienical management, and also manage water purification quality performance.

The related art of Korean Patent Application Publication No. 10-2017-0005264 discloses a water treatment apparatus including: a water quality measuring part which measures the quality of water stored in the purification tank and generates water quality information; a circulation channel which provides a path resupplying the stored purified water in the purification tank to the filtration part; and a controlling part which drains the stored purified water in the purification tank depending on the water quality information or resupplies the purified water to the filtration part through the circulation channel.

### DISCLOSURE

### Technical Problem

An aspect of the present disclosure is directed to providing a water dispensing device capable of measuring a water quality more accurately.

Yet another aspect of the present disclosure is directed to providing a water dispensing device capable of preventing product malfunction due to a false signal of a sensor value in advance and increasing sensor reliability by compensating for abnormal signals with a processing algorithm when a sensor value of a water quality measuring unit is output.

Yet another aspect of the present disclosure is directed to providing a water dispensing device capable of automatically sensing water quality abnormalities and component abnormalities such as a sensor and hygienically managing the same.

Yet another aspect of the present disclosure is directed to providing a water dispensing device capable of improving sensing accuracy and efficiency by a water pipe configuration and a rinse operation for sensor publicization.

### Technical Solution

A water dispensing device according to an embodiment of the present disclosure includes: a water supply channel through which raw water supplied from a water supply source flows; a filter which generates purified water by filtering the raw water supplied through the water supply channel; a purified water channel through which the purified water passed through the filter flows; and a water quality measuring unit which is connected to the purified water channel to measure a water quality of the purified water, wherein the water quality measuring unit includes a turbidity sensor, and the turbidity sensor collects N pieces of turbidity data during a first sensing time, outputs a sensing result on the basis of the N pieces of turbidity data when there is no abnormality in the collected N pieces of turbidity data, and re-collects the turbidity data to determine whether there is an abnormality when there is an abnormality in the collected N pieces of turbidity data.

The turbidity sensor may re-collect turbidity data during a second sensing time that is longer than the first sensing time.

The turbidity sensor may: calculate an average value of the N pieces of turbidity data; calculate an error ratio between the N pieces of turbidity data and the average value; and determine whether there is an abnormality when an absolute value of the calculated error ratio has a value exceeding a reference value.

The water dispensing device according to an embodiment of the present disclosure may further include a control unit that stops operation of at least one of a compressor or a pump when there is an abnormality as a result of determining the abnormality after re-collecting the turbidity data.

After the turbidity data are re-collected, when there is no abnormality as a result of determining the abnormality, a sensing result based on the re-collected turbidity data may be output.

When no normal data is collected until a third sensing period that is longer than the first sensing period, the control unit may stop operation of at least one of the compressor or the pump.

The turbidity sensor may collect the turbidity data in a state where at least one of the compressor or the pump is stopped from operating.

The control unit may determine that there is an abnormality with the turbidity sensor when there is an abnormality in data collected in a state where at least one of the compressor or pump is stopped from operating, and may determine that there is an abnormality with at least one of the compressor or the pump when there is no abnormality in the data collected in the state where at least one of the compressor or the pump is stopped from operating.

The control unit may automatically connect a service of a component determined as an abnormality.

The sensing result may be an average value of the N pieces of turbidity data.

The water dispensing device according to an embodiment of the present disclosure may further include a sensing channel diverged from the water supply channel and through which the raw water flows, wherein the water quality measuring unit may measure the water quality of the purified water when the purified water enters through the purified water channel and may measure the water quality of the raw water when the raw water enters through the sensing channel.

The water quality measuring unit may measure the water quality of the purified water after performing a rinse operation in which the purified water passes through at least once when measuring the water quality of the raw water.

The water dispensing device according to an embodiment of the present disclosure may further include a switching valve for supplying the raw water to the water supply channel or the sterilization channel, and a sensing valve for opening and closing the sensing channel.

The water dispensing device according to an embodiment of the present disclosure may further include: a water outlet through which the purified water is discharged; a water outlet channel guiding the purified water to the water outlet; and a drainage channel diverged from the water outlet channel between the water quality measuring unit and the water outlet through which the raw water or the purified water is drained.

The water dispensing device according to an embodiment of the present disclosure may further include a water outlet valve selectively supplying the raw water or the purified water to the water outlet channel and the drainage channel.

The water dispensing device according to an embodiment of the present disclosure may further include: a hot water channel diverged from the purified water channel at one side; a hot water module provided in the hot water channel to heat purified water passing through the hot water channel; a cold water channel diverged from the purified water channel at one side; and a cold water module provided in the cold water channel to cool purified water passing through the cold water channel.

The water dispensing device according to an embodiment of the present disclosure may further include a drainage pump disposed in the drainage channel.

The turbidity sensor may irradiate light to the raw water or a portion of the purified water and sense turbidity based on a pattern of scattered light received.

### Advantageous Effects

According to at least one of the embodiments of the present disclosure, a water quality can be measured more accurately.

According to at least one of the embodiments of the present disclosure, when a sensor value of a water quality measuring unit is output, a processing algorithm for an abnormal signal is used to compensate, thereby preventing product malfunction due to a false signal of the sensor value in advance and increasing sensor reliability.

According to at least one of the embodiments of the present disclosure, water quality abnormalities and component abnormalities such as a sensor can be automatically sensed, thereby enabling hygienical management.

According to at least one of the embodiments of the present disclosure, the sensing accuracy and efficiency can be improved by a water pipe configuration and a rinse operation for sensor publicization.

Various other benefits will be described directly or implicitly in the detailed description according to the embodiments of the present disclosure to be described later.

### DESCRIPTION OF DRAWINGS

FIG. 1 is a block diagram illustrating the main configuration of a water dispensing device according to an embodiment of the present disclosure.

FIG. 2 is a conceptual diagram illustrating a water dispensing device according to an embodiment of the present disclosure.

FIGS. 3 to 5 are diagrams for reference in describing the operation of the water dispensing device of FIG. 2.

FIG. 6 is a conceptual diagram illustrating a water dispensing device according to an embodiment of the present disclosure.

FIG. 7 is a flowchart of an operation method of a water dispensing device according to an embodiment of the present disclosure.

FIG. 8 is a flowchart of an operation method of a water dispensing device according to an embodiment of the present disclosure.

FIGS. 9 to 12 are diagrams for reference in describing an operation method of a water dispensing device according to an embodiment of the present disclosure.

### MODE FOR DISCLOSURE

Hereinafter, the exemplary embodiments of the present disclosure are described in detail with reference to the accompanying drawings. The present disclosure may, however, be embodied in many different forms and should not be construed as being limited to the embodiments set forth herein.

In order to clearly and briefly describe the present disclosure, the parts irrelevant to the description are omitted in the drawings. The same reference numerals are used to designate the same or similar parts throughout the specification.

With respect to constituents used in the following description, the suffixes “module” and “unit” are merely given in consideration of only facilitation of description and do not have any special importance or role. Accordingly, the “module” and the “unit” may be used interchangeably.

In addition, it will be understood that, although the terms “first”, “second”, etc. may be used herein to describe various elements, these elements should not be limited by these terms. These terms are only used to distinguish one element from another element.

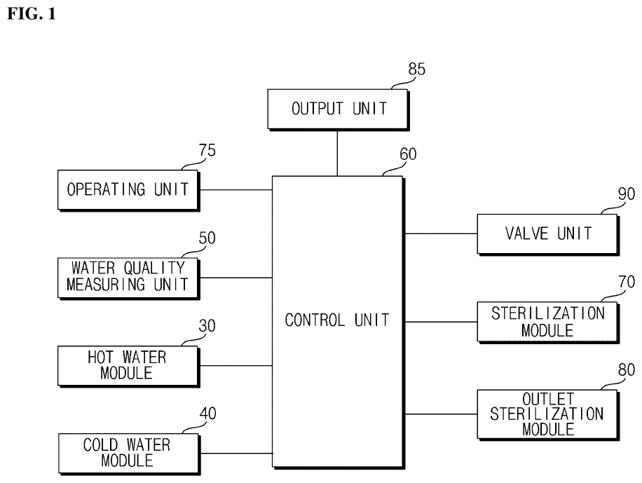

FIG. 1 is a block diagram illustrating the main configuration of a water dispensing device according to an embodiment of the present disclosure.

Referring to FIG. 1, the water dispensing device according to an embodiment of the present disclosure includes a water quality measuring unit 50. The water quality measuring unit 50 may include a turbidity sensor. According to an embodiment of the present disclosure, turbidity (contamination level) may be sensed through optical sensing. The optical sensing method for measuring turbidity uses a transmitted light method and a scattered light method. The transmitted light method senses turbidity by irradiating light on a fluid, receiving light transmitted through the fluid, and processing data. The scattered light method senses turbidity by receiving scattered light and processing data, and is subdivided according to a method of generating scattered light and a method of processing the data received by light. The turbidity sensor according to an embodiment of the present disclosure may irradiate light to the raw water or a portion of the purified water and sense turbidity based on a pattern of scattered light received. According to an embodiment of the present disclosure, particles and microorganisms are distinguished by patterning the intensity and movement of light scattered by microorganisms, and the types of water quality/hygiene standard indicator microorganisms are distinguished by big data processing. In addition, the sensed microbial concentration values and safety indicators are provided through a display so that a user may intuitively check the same.

In addition, the water quality measuring unit 50 may utilize water quality measurement sensors such as the turbidity sensor, a microbial sensor, and a TDS sensor to sense water quality contamination of water within a channel. The water quality measuring unit 50 may include at least one of the turbidity sensor, a microorganism detection sensor, a chlorine sensor, a total dissolved solids (TDS) sensor, or a BOD (biochemical oxygen demand) sensor, and may measure at least one of turbidity, microorganisms, residual chlorine, TDS, or dissolved oxygen of entering water. At least one of the sensors provided in the water quality measuring unit 50 may be a common sensor that measures the water qualities of both raw water and purified water.

In this specification, the entering of purified water or raw water in the water quality measuring unit 50 does not mean only that purified water or raw water enters inside the water quality measuring unit 50. For example, a portion of the purified water or raw water may be sampled and discharged after measuring the water quality inside the water quality measuring unit 50. In addition, at least some of the sensors provided in the water quality measuring unit 50 may measure the water quality of a flowing liquid. In this case, the entering of purified water or raw water in the water quality measuring unit 50 may mean that at least a portion of the purified water or raw water passes through the sensible section of the water quality measuring unit 50.

For example, in the case where the turbidity sensor has an internal chamber, the turbidity sensor may measure water quality by irradiating light on the filled water and receiving a scattered light pattern when water enters the internal chamber connected to a channel 20 and is filled. In addition, the water in the internal chamber may be discharged after measuring the water quality. Alternatively, a light source unit and a light receiving unit of the turbidity sensor may be disposed in a specific channel section (for example, a section after the purified water channel 20 and a sensing channel 12 are joined) to irradiate light to purified water or raw water passing through the specific channel section and receive a scattered light pattern.

The water dispensing device according to an embodiment of the present disclosure may measure the water quality of raw water and purified water using the common sensor for the same measurement item, such as turbidity. The turbidity sensor measures the turbidity of raw water and purified water, and transmits the sensing data to a control unit 60. The control unit 60 may control other configurations of the water dispensing device based on sensing data from the water quality measuring unit 50 such as the turbidity sensor.

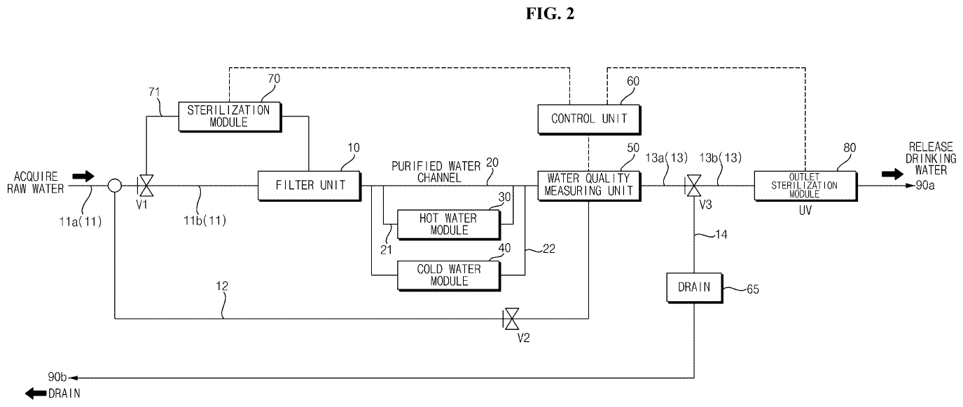

The water dispensing device includes a filter 10 (FIG. 2) that filters the raw water supplied from a water supply source to produce purified water. The filter 10 is for purification of supplied raw water and filters out various impurities and harmful substances contained in the raw water. One or more filters 10 may be provided, and in a case where a plurality of filters are provided, the filter 10 may be configured to combine filters with various functions. For example, the filter 10 may be provided in three pieces, and may include a pre-carbon filter and a post-carbon filter, and a membrane filter or a hollow fiber membrane filter disposed between the pre-carbon filter and the post-carbon filter. Alternatively, the filter 10 may be configured of a pre-carbon filter and a UF composite filter.

The purified water purified from the filter 10 flows to a storage tank or the channel 20 (FIG. 2). Since water stored in the storage tank becomes a better environment for microbial growth over time, it is more desirable to flow directly through the channel 20. The purified water that has passed through the filter 10 flows into the purified water channel 20.

In addition, the water dispensing device includes a valve unit 90 having valves for controlling the flow of water. The valve unit 90 may include a plurality of valves V1, V2, V3 to be described below.

When the water quality measuring unit 50 measures the water quality of the raw water, the control unit 60 may control the valve unit 90 to perform a rinse operation in which the purified water passes through the water quality measuring unit 50 at least once.

The water quality measuring unit 50 may minimize the influence of raw water on the measurement of the water quality of the purified water by measuring the water quality of the purified water after performing the rinse operation. Accordingly, a single water quality sensor of the same type may efficiently and accurately sense both raw water and purified water.

In the case of tap water and purified outlet water, the water quality contamination level is normally low. In order to measure a low-concentration contamination level, it is important to minimize the occurrence of deviation in the measurement values. According to an embodiment of the present disclosure, compared to a technology that measures raw water and outlet water separately, a channel configuration and control capable of simultaneously measuring raw water and outlet water using one sensor is provided, thereby minimizing an increase in material costs and enabling a compact product configuration.

Since the tap water quality (raw water) and water purifier quality (purified water) are mostly in a low-concentration section, it is most important to minimize measurement deviation between devices by comparison with a single sensor in order to clearly express the difference in performance between raw water and outlet water.

In addition, the water dispensing device may include a hot water module 30 and a cold water module 40 for providing hot and cold water. The hot water module 30 heats purified water and then discharges the same toward a water outlet 90a (FIG. 2). The cold water module 40 cools the purified water and then discharges the same toward the water outlet 90a.

In addition, the water dispensing device further includes an operating unit 75 and an output unit 85.

The operating unit 75 may include one or more buttons for receiving user input. For example, the operating unit 75 may be provided with a touch panel and may include a capacity button for selecting a water outlet capacity, a hot water button for selecting hot water and further selecting the temperature of hot water to be released, a purified water button for selecting purified water, a cold water button for selecting cold water, and other function buttons.

The output unit 85 may be provided with a display device such as a display (not shown) or a light emitting diode (LED) (not shown). For example, the output unit 85 may display information such as the driving state of the water dispensing device, the operation state related to the occurrence of an error, or the contamination level of water.

The output unit 85 may be provided with an audio device such as a speaker (not shown) or a buzzer (not shown). For example, the output unit 85 may output a sound effect regarding the driving state of the water dispensing device and may output a predetermined warning sound when an error occurs.

In addition, the water dispensing device may include modules for hygiene. For example, the water dispensing device includes a sterilization module 70 using high temperature water. In addition, the water dispensing device includes an outlet sterilization module 80 for sterilizing the water outlet 90a where contamination is highly likely to occur.

The sterilization module 70 may sterilize bacteria growing in water by instantly heating the water to a high temperature. In addition, the control unit 60 may operate the sterilization module 70 and circulate the sterilizing water (high temperature water) from the sterilization module 70 to another channel to sterilize the channel. The control unit 60 may control the high temperature water released from the sterilization module 70 to move to different channel regions based on the water quality data measured by the water quality measuring unit 50 and perform a sterilization operation for each channel region.

The outlet sterilization module 80 irradiates ultraviolet rays toward the water outlet 90a to remove bacteria or viruses. The outlet sterilization module 80 may include at least one ultraviolet (UV) lamp or at least one UV light emitting diode (LED).

The outlet sterilization module 80 may be driven periodically under the control of the control unit 60. In addition, the outlet sterilization module 80 may be driven for a predetermined period of time before water outlet. More preferably, the control unit 60 may improve efficiency by driving the outlet sterilization module 80 only when necessary based on water quality data measured by the water quality measuring unit 50. For example, the control unit 60 may control the outlet sterilization module 80 based on the water quality measurement results of purified water.

The control unit 60 may be connected to each component provided in the water dispensing device. For example, the control unit 60 can transmit and/or receive signals between each component provided in the water dispensing device and control the overall operation of each component.

The control unit 60 may include at least one processor, and may control the overall operation of the water dispensing device using the processor included therein. Herein, the processor may be a general processor such as a central processing unit (CPU). The processor may be a dedicated device such as an ASIC, or may be any of other hardware-based processors.

The control unit 60 may perform various calculations based on data received through the water quality measuring unit 50 including various sensors such as the turbidity sensor 51. In addition, the control unit 60 may store data received through the water quality measuring unit 50 in a memory (not shown).

The water quality measuring unit 50 may measure water quality and output the same to the control unit 60. The control unit 60 may be controlled to perform a feedback operation in response to water quality measurement data of raw water and/or purified water. Alternatively, the water quality measuring unit 50 may directly determine a contamination level and transmit the same to the control unit 60, and the control unit 60 may control other components to perform appropriate feedback operations based on the received contamination level.

The control unit 60 may control the output unit 85 to recognize the contamination state of raw water and/or purified water and provide a user with information on a cleaning alarm or filter replacement cycle.

In addition, the control unit 60 may sense in advance any odor that may occur depending on the contamination level of raw water and/or purified water, and operate the automatic washing/sterilization logic through the sterilization module 70 and the outlet sterilization module 80 before the customer feels so. Accordingly, the convenience and hygiene of use for non-professional users may be improved.

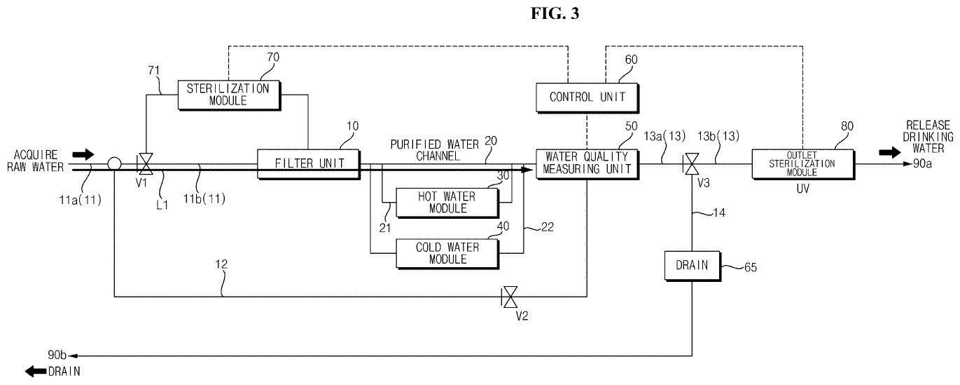

FIG. 2 is a conceptual diagram illustrating a water dispensing device according to an embodiment of the present disclosure. FIGS. 3 to 5 are diagrams for reference in describing the operation of the water dispensing device of FIG. 2.

Referring to FIG. 2, the water dispensing device includes the water supply channel 11 through which raw water supplied from the water supply source flows, and the filter unit 10 that filters the raw water supplied to the water supply channel 11 to produce purified water.

The purified water that has passed through the filter unit 10 flows through the purified water channel 20 toward the water outlet 90a. The purified water that has passed through the filter unit 10 may enters the water quality measuring unit 50. The water quality measuring unit 50 may measure the water quality of purified water when the purified water enters.

In addition, the sensing channel 12 is diverged from the water supply channel 11, so that raw water may directly enter the water quality measuring unit 50 through the sensing channel 12. The water quality measuring unit 50 may measure the water quality of raw water when the raw water enters.

According to an embodiment of the present disclosure, a water supply valve V1 that controls the water supply to the filter unit 10 and the purified water channel 20 may be disposed in the water supply channel 11. The water supply valve V1 may open and close the purified water channel 20. When the water supply valve V1 is opened, raw water may be purified through the filter unit 10 along a first line L1 of FIG. 3, and purified water may enter the water quality measuring unit 50 through the purified water channel 20.

The water supply channel 11 may include a first water supply channel 11a connecting the water supply source and the water supply valve V1, and a second water supply channel 11b connecting the water supply valve V1 and the filter 10.

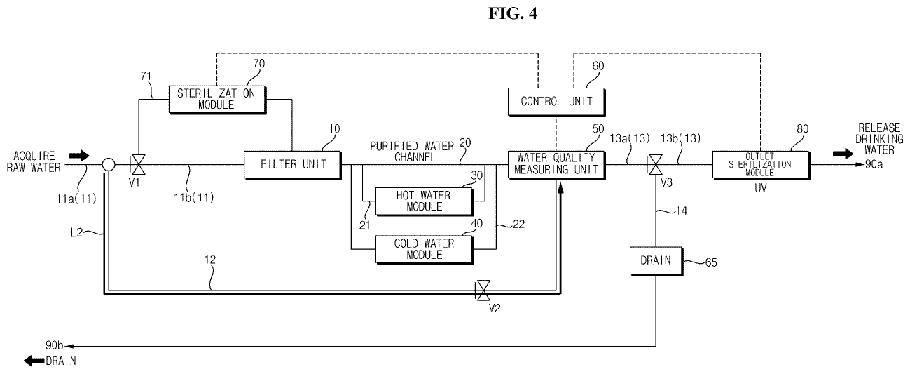

In addition, one end of the sensing channel 12 may be connected to the first water supply channel 11a, and the other end thereof may be connected to the water quality measuring unit 50. A sensing valve V2 that opens and closes the sensing channel 12 may be disposed in the sensing channel 12. When the sensing valve V2 is opened, raw water may directly enter the water quality measuring unit 50 through the sensing channel 12 along a second line L2 of FIG. 4.

Referring to FIG. 2, the water dispensing device may further include: the water outlet 90a through which the purified water is discharged; a water outlet channel 13 guiding the purified water to the water outlet 90a; a drainage channel 14 diverged from the water outlet channel 13 between the water quality measuring unit 50 and the water outlet 90a through which the raw water or the purified water is drained; and a water outlet valve V3 selectively supplying the raw water or the purified water to the water outlet channel 13 and the drainage channel 14.

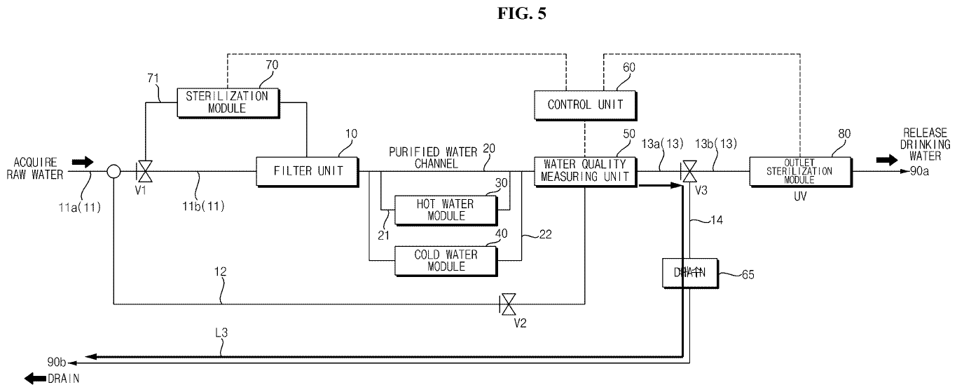

The water outlet valve V3 may, under the control of the control unit 60, divert water for which water quality measurement is completed to a drain 90b and the water outlet 90a. When the drainage operation is performed, the water for which water quality measurement is completed is allowed to flow in the drainage channel 14 along a third line L3 of FIG. 5. By draining and washing raw water, the raw water may be prevented from being released as drinking water.

The water outlet channel 13 may include a first water outlet channel 13a connecting the water quality measuring unit 50 and the water outlet valve V3, and a second water outlet channel 13b connecting the water outlet valve V3 and the water outlet 90a.

On the side of the water outlet 90a through which the purified water is discharged, the outlet sterilization module 80 that irradiates ultraviolet rays to the water outlet 90a is disposed. The outlet sterilization module 80 may sterilize a water outlet space and residual water. The control unit 60 may operate the outlet sterilization module 80 for a predetermined time based on the water quality data measured by the water quality measuring unit 50.

Referring to FIG. 2, a sterilization channel 71 has one side diverged from the water supply channel 11 and the other side connected to the filter 10, and the sterilization module 70 that heats water passing through the sterilization channel 71 is disposed in the sterilization channel 71. The control unit 60 may operate the sterilization module 70 for a predetermined time based on the water quality data measured by the water quality measuring unit 50.

According to an embodiment of the present disclosure, the water supply valve V1 may be a switching valve that selectively supplies the raw water to the water supply channel 11 or the sterilization channel 71.

Referring to FIG. 2, the water dispensing device may further include: a hot water channel 21 diverged from the purified water channel 20 at one side; the hot water module 30 provided on the hot water channel 21 to heat purified water passing through the hot water channel 21; a cold water channel 22 diverged from the purified water channel 20 at one end side; and the cooling module 40 provided on the cold water channel 22 to cool purified water passing through the cold water channel 22.

Referring to FIG. 2, the hot water channel 21 and the cold water channel 22 may be joined again to the purified water channel 2. Alternatively, the hot water channel 21 and the cold water channel 22 may be joined to the water outlet channel 13.

According to an embodiment of the present disclosure, a drainage pump 65 may be disposed in the drainage channel 14. After measuring the water quality, when the drainage pump 65 operates, the water whose water quality has been measured may be discharged to the outside at a faster speed.

In addition, the drainage pump 65 may operate while the sterilization operation for each channel region is performed. Accordingly, the high temperature water may be discharged to the outside more quickly after sterilization. In particular, when the water outlet channel 13 connected to the cock on the water outlet 90a is also sterilized, a portion of the high temperature water is discharged toward the water outlet 90a, but a large amount of the high temperature water may be discharged toward the drain 90b. Accordingly, the occurrence of safety accidents, discomfort to users, and the inconvenience of users having to handle a large amount of hot water may be prevented as a large amount of hot water is discharged through the water outlet 90a.

The water quality measuring unit 50 measures the water quality of the purified water when the purified water enters through the purified water channel 20, and measures the water quality of the raw water when the raw water enters through the sensing channel 12.

When the water quality measuring unit 50 measures the water quality of the raw water, the water quality of the purified water may be measured after performing a rinse operation in which the purified water passes through at least once. As such, the sensing accuracy and efficiency may be improved by a water pipe configuration and a rinse operation for sensor publicization.

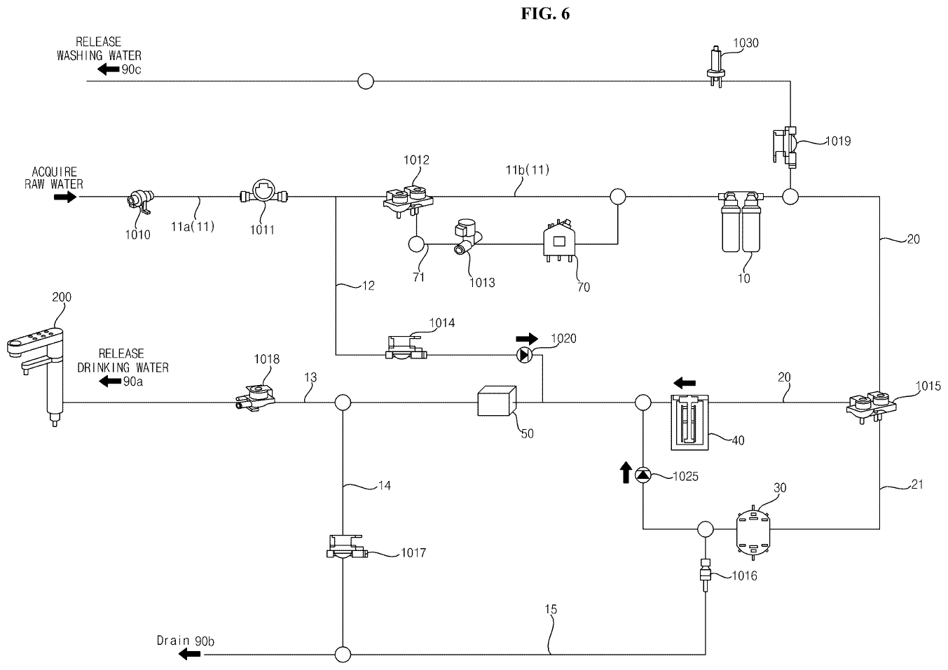

FIG. 6 is a conceptual diagram illustrating a water dispensing device according to an embodiment of the present disclosure, and illustrates a water pipe of the water dispensing device according to an embodiment of the present disclosure.

The water dispensing device according to an embodiment of the present disclosure may correspond to various water treatment apparatuses and purification apparatuses, such as water purifiers and refrigerators where water enters from the outside, purify the entered water, and then discharge the purified water.

As an example, the water dispensing device may be provided with an under sink type water purifier in which at least a portion is disposed in the lower space of a sink.

Referring to FIG. 6, the water dispensing device according to an embodiment of the present disclosure may include a water outlet unit 200 installed so that at least a portion thereof is exposed to the outside of the sink and a remaining main body unit installed on the inside of the sink.

The water dispensing device includes the water supply channel 11 that guides raw water supplied from the outside to the inside, the filter 10 that purifies the raw water supplied along the water supply channel 11 into purified water, and the purified water channel 20 that causes purified water passing through the filter 10 to flow toward the water outlet unit 200.

The water supply channel 11 connects an external water supply source and the filter 10. Through the water supply channel 11, raw water supplied from the external water supply source may be supplied to the filter 10.

As described above, water (raw water) supplied to the filter 10 is purified into purified water by passing through the filter 10. At least one of the filters 10 may be provided. For example, a plurality of filters 10 may be provided. Accordingly, water passing through the water supply channel 11 may be purified into cleaner water by passing through the plurality of filters 10.

In addition, purified water passing through the filter 10 may flow through the purified water channel 20 toward the water outlet unit 200 exposed on the outside of the sink 10.

To this end, one end of the purified water channel 20 is connected to the filter 10, and the other end is connected to the water outlet unit 200. In the purified water channel 20, at least one of the cold water channel 22, the hot water channel 21, or a washing water channel 90c may be diverged.

In FIG. 6, the cold water channel 22 is integrated into the purified water channel 20, and an example is illustrated in which the hot water channel 21 and the washing water channel 90c are diverged from the purified water channel 20.

One end of the purified water channel 20 is connected to the filter 10, and water passing through the filter 10 flows toward the water outlet unit 200 through the connected water outlet channel 13. The water outlet unit 200 includes the water outlet 90a and may take out purified water.

In addition, water diverged into the washing water channel 90c may be supplied to a washing water outlet in the state of sterilized water while passing through a washing water module 1030 provided on the washing water channel 90c. In the case where the water outlet unit 200 includes a plurality of water outlets, the washing water outlet may also be formed in the water outlet unit 200 depending on an embodiment.

A pressure reducing valve 1010 for adjusting the flow rate of water supplied to the filter 10 may be installed in the water supply channel 11.

In addition, at least one of a flow sensor 1011 for sensing the flow rate of water, an inlet valve for adjusting the flow rate of water or regulating the flow of water, or a flow rate sensor (not shown) for sensing the flow rate of water may be installed in the water supply channel 11 or the purified water channel 20.

In addition, separate opening/closing valves for regulating water flow in each of the purified water channel 20, the hot water channel 21, and the washing water channel 90c may be installed. For example, a washing water valve 1019 may be disposed in the washing water channel 90c.

Alternatively, a cold/hot/purified water valve 1015 that may selectively supply purified water to the purified water channel 20 and the hot water channel 21 may be installed at a diverge point of the purified water channel 20 and the hot water channel 21.

In addition, a device 1025 for safety such as backflow prevention may be installed in the hot water channel 21. In addition, a safety valve 1016 for steam discharge may be installed in the hot water module 30. The steam from the hot water module 30 may be drained toward the drain 90b through a connected channel 15.

A water outlet valve 1018 may be disposed in the water outlet channel 13 to supply or block purified water, cold water, and hot water flowing toward the water outlet unit 200 to the water outlet unit 200.

In addition, the drainage channel 14 is diverged from the water outlet channel 13, and a drain valve 1017 is disposed in the drainage channel 14 so that purified water, cold water, hot water, and raw water may be discharged toward the drain 90b.

As an example, the water outlet valve 1018 and the drain valve 1017 may be provided as a three-way valve having one inlet, a first outlet and a second outlet that are selectively opened, and an actuator that selectively opens and closes the two outlets. In this connection, the first outlet may be connected to the water outlet unit 200, and the second outlet may be connected to the drain 90b.

Raw water is supplied through the water supply channel 11 connected to the water supply source such as a tap water pipe, a water tank, or an underground water pipe. The pressure reducing valve 1010 is installed in the water supply channel 11, and the raw water is reduced to a set pressure while passing through the pressure reducing valve 1010.

In addition, the raw water that passes through the filter 10 becomes purified with foreign substances removed. The purified water flows along the purified water channel 20. In addition, the purified water may be diverged into cold-purified water and hot water.

First, the purified water diverged into the cold-purified water is diverged again into cold water and purified water. Depending on the operation of the cold water module 40 corresponding to the purified water or cold water selection manipulation of a user, the purified water or cold water may be supplied to the user through the water outlet unit 200.

When a user requests the release of cold water, purified water passes through a cooling coil inside the cold water module 40. The water flowing along the cooling coil exchanges heat with the coolant inside the cold water module 40 and is cooled into cold water. To this end, the coolant is continuously cooled to maintain a set temperature. For reference, a compressor may be driven to cool the coolant. The driving of the compressor may be determined by a cold water temperature sensor provided inside the cold water module 40. Accordingly, the coolant may always maintain a set temperature, and to this end, the driving of the compressor may be adjusted. The compressor is an inverter compressor, and its frequency is adjusted in response to the required load, and its cooling capacity may be adjusted. In other words, the compressor may be driven by inverter control and cool the coolant at optimal efficiency.

When a user requests the release of hot water, the purified water may be heated to a set temperature while passing through the hot water module 30. The hot water module 30 may be heated by induction heating, and to this end, the output of the working coil included in the hot water module 30 may be adjusted. The purified water passing through the hot water module 30 may be heated to a set temperature. The heated hot water passes through the hot water module 30 and flows toward the water outlet unit 200.

The sterilization channel 71 has one side diverged from the water supply channel 11 and the other side connected to the filter 10. In the sterilization channel 71, a sterilization module 70 that heats water passing through the sterilization channel 71 and a flow rate adjustment valve 1013 that controls the flow rate of the sterilization channel 71 may be disposed.

At a location where the sterilization channel 71 is diverged from the water supply channel 11, a feed valve 1012 that selectively supplies the raw water to the water supply channel 11 or the sterilization channel 71 may be disposed.

The water supply channel 11 may include the first water supply channel 11a connecting the water supply source and the feed valve 1012, and the second water supply channel 11b connecting the feed valve 1012 and the filter 10.

The sensing channel 12 described above may be diverged in the first water supply channel 11a at a front end of the feed valve 1012. A sensing valve 1014 that opens and closes the sensing channel 12 and a backflow prevention device 1020 that prevents backflow of raw water may be disposed in the sensing channel 12.

The control unit 60 may control the feed valve 1012 to be closed and the sensing valve 1014 to be opened so that raw water is supplied to the water quality measuring unit 50 through the sensing channel 1014.

After measuring the water quality of raw water, the control unit 60 closes the water outlet valve 1018 and opens the drain valve 1017 to discharge the raw water measured by the water quality measuring unit 50 toward the drain 90b.

The control unit 60 may control the feed valve 1012 to open toward the purified water channel 20 and the sensing valve 1014 to close so that purified water is supplied to the water quality measuring unit 50.

In addition, the control unit 60 closes the water outlet valve 1018 and opens the drain valve 1017 to perform a rinse operation by controlling the purified water passing through the water quality measuring unit 50 to be discharged toward the drain 90b.

Thereafter, the control unit 60 may measure the water quality of purified water by supplying purified water to the water quality measuring unit 50 with the same valve control. Accordingly, the influence of the raw water may be removed and the water quality of the purified water may be accurately measured in the same water quality measuring unit 50.

As described above, the water quality measuring unit 50 includes the turbidity sensor. The turbidity sensor may irradiate light to the raw water or a portion of the purified water and sense turbidity based on a pattern of scattered light received. For example, the turbidity sensor may sense the scattered light from a visible light laser source that is reflected and dispersed by floating substances in water and output the same as a signal value.

Scattered light increases proportionally with the amount of particles in the fluid, but may be affected by external noise generated by the behavior of the particles, the fluid state (physical environment such as the generation of bubbles or vortices), or the external environment such as vibration. Regardless of the amount of particles, external noise that may be added to the scattered light signal occurs, causing the signal value to be measured in an exaggerated/understated manner in the turbidity sensor.

Hereinafter, a compensation algorithm that filters and processes abnormal values of sensor signal values to prevent product malfunction due to such noise in advance will be described in detail. According to an embodiment of the present disclosure, product malfunction due to a false signal of a sensor value may be prevented, thereby reducing the occurrence rate of customer claims or product defects.

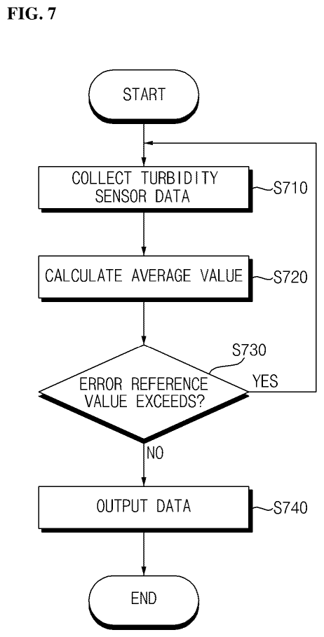

FIG. 7 is a flowchart of an operation method of a water dispensing device according to an embodiment of the present disclosure.

Referring to FIG. 7, the turbidity sensor of the water quality measuring unit 50 senses the turbidity of purified water passing through the filter 10 in real time and collects turbidity data (S710). Alternatively, the turbidity sensor may sense the water quality of raw water bypassed through the sensing channel 12 and collect turbidity data. The turbidity sensor collects N pieces of turbidity data during a first sensing time (for example, 1 second, 5 seconds, etc.).

The turbidity sensor determines whether the collected turbidity data is abnormal (S730), and depending on the determination result, outputs the data to the control unit 60 (S740), or re-collects turbidity data to acquire data without abnormalities (S710).

The determination of whether the collected turbidity data is abnormal (S730) may be performed by comparing each N piece of the turbidity data with a fixed reference value. However, since turbidity data have various values depending on water quality, there are limitations in using fixed reference values.

Accordingly, more preferably, the turbidity sensor may calculate an average value of the N pieces of turbidity data (S720) and compare each N piece of the turbidity data with the calculated average value to determine whether there is an abnormality (S730).

The turbidity sensor may calculate an error ratio between the N pieces of turbidity data and the average value, and determine that there is an abnormality when the absolute value of the calculated error ratio exceeds a reference value (for example, 20%). In other words, when even one value deviates from the reference range set based on the average value, the data collected in the first sensing period in which the data was collected may not be used at all and may be discarded.

When there is no abnormality in the collected N pieces of turbidity data (S730), the turbidity sensor outputs the sensing result based on the N pieces of turbidity data to the control unit 60 (S740). For example, the turbidity sensor may output an average value of the N pieces of turbidity data collected in the first sensing period as a sensing result.

When there is an abnormality in the N pieces of data collected (S730), the turbidity data may be re-collected to determine whether there is an abnormality (S710 to S730).

As such, when the sensor value is output, the data from the sensing period including the abnormal value is discarded, and only the sensing value from when all pieces of data is re-collected and decided to be valid is used, thereby improving the sensing accuracy.

In addition, it is possible to prevent product malfunctions due to a false signal of a sensor value and increase sensor reliability. In addition, even when low-cost sensors are used, a certain level of accuracy may be secured.

According to an embodiment, when there is an abnormality in the data (S730) and data is re-collected (S710), the turbidity sensor may re-collect turbidity data during a second sensing time that is longer than the first sensing time. Accordingly, data verification of the turbidity sensor may be made more rigorous.

The control unit 60 may stop the operation of other components that may cause vibration for more accurate sensing. For example, the control unit 60 may stop the operation of at least some of the compressor or various pumps depending on the configuration of the water dispensing device.

According to an embodiment of the present disclosure, the control unit 60 may stop operation of at least one of the compressor or the pump when an abnormality is found after re-collecting the turbidity data. For example, the cold water module 40 may include a compressor, and the control unit 60 may stop the operation of the cold water module 40 during the sensing time. For example, the pump may be the drainage pump 65. Alternatively, a pump may be provided depending on the type of device provided with the water dispensing device, the water piping structure, and the environment in which the water dispensing device is disposed. The control unit 60 may stop the operation of at least some of the pumps during the sensing time.

The control unit 60 may stop operation of vibrating components such as a compressor when re-collecting data one or more times to prevent external noise caused by vibration from affecting the data.

In another embodiment, the control unit 60 may stop operation of at least one of the compressor or the pump when no abnormal data is collected for a third sensing period that is longer than the first sensing period.

The turbidity sensor may output a sensing result based on the re-collected turbidity data when no abnormality is found as a result of determining the abnormality after re-collecting the turbidity data.

The turbidity sensor may collect turbidity data in a state where at least one of the compressor or the pump is stopped from operating.

The control unit 60 may determine that the turbidity sensor is abnormal when there is an abnormality in the data collected in a state where at least one of the compressor and the pump is stopped from operating.

The control unit 60 may determine that at least one of the compressor or the pump is abnormal when there is no abnormality in the data collected in a state where at least one of the compressor or the pump is stopped from operating.

In addition, the control unit 60 may control the output unit 85 to output abnormal information of a component determined to be abnormal. In addition, when the water dispensing device is provided with a communication module (not shown), the control unit 60 may automatically connect the service of the component determined as abnormal.

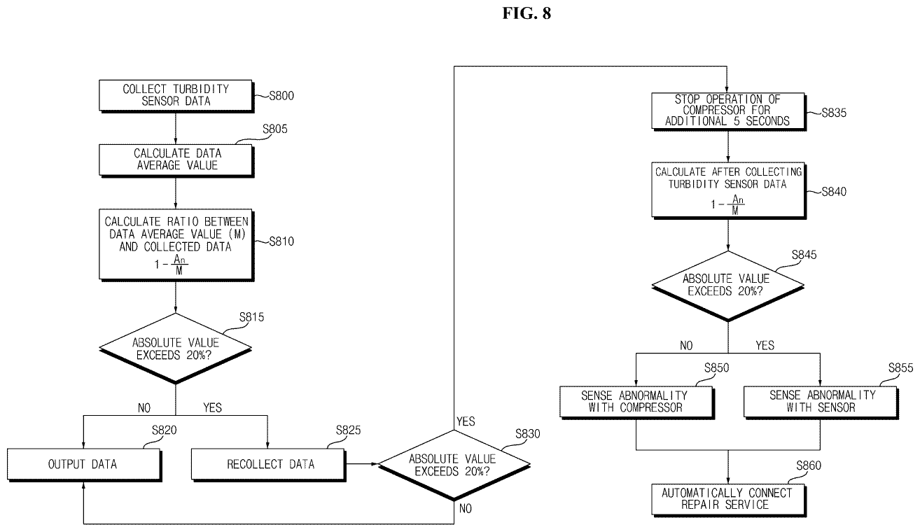

FIG. 8 is a flowchart of an operation method of a water dispensing device according to an embodiment of the present disclosure.

Referring to FIG. 8, the turbidity sensor of the water quality measuring unit 50 senses the turbidity of the incoming water in real time and collects turbidity data (S800). The turbidity sensor collects N pieces of turbidity data (A1, A2 . . . An) during a first sensing time (for example, 1 second or 5 seconds).

The turbidity sensor may calculate an average value (M) of the N pieces of turbidity data (S805), and calculate an error ratio (1−An/M, n=1,2, . . . , N) between the calculated average value (M) and each piece of collected turbidity data (S810).

The turbidity sensor may determine whether there is an abnormality by comparing the absolute value of the error ratio with the reference value (S815). For example, the reference value set for the error ratio may be 20%.

The turbidity sensor may calculates an error ratio between the N pieces of turbidity data and the average value (S810), and determine that there is an abnormality (S815) when the absolute value of the calculated error ratio exceeds a reference value (for example, 20%).

When there is no turbidity data exceeding the reference value (S815), the turbidity sensor may output data to the control unit 60 (S820). When there is turbidity data exceeding the reference value (S815), the turbidity sensor may re-collect data (S825).

Re-collected data may also be verified in the same way. Accordingly, the average value of the re-collected data is calculated, and when the error ratios of the re-collected data and the average value do not exceed the reference value (S830), the turbidity sensor may output data to the control unit 60 (S820).

Additionally, the control unit 60 may stop the operation of the compressor and/or pump for an additional re-collection time (for example, 5 seconds) (S835). The turbidity sensor may collect data during an additional re-collection time, and calculate an average value of the re-collected turbidity data and an error ratio between the re-collected turbidity data and the average value (S840). In addition, the turbidity sensor may determine whether there is an abnormality by comparing the absolute value of the error ratio with a reference value (S845).

When there is no abnormality in the data collected in a state where at least one of the compressor or the pump is stopped from operating (S835), the turbidity sensor and/or the control unit 60 may determine that at least one of the compressor or the pump is abnormal (S850).

The turbidity sensor and/or the control unit 60 may determine that the turbidity sensor is abnormal (S855) when there is an abnormality in the collected data (S845) in a state where at least one of the compressor or the pump is stopped from operating (S835).

In addition, the control unit 60 may control the output unit 85 to output abnormal information of a component determined to be abnormal. In addition, when the water dispensing device is provided with a communication module (not shown), the control unit 60 may automatically connect the service of the component determined as abnormal (S860). For example, when at least one of the compressor or the pump is determined to be abnormal (S850), information on the abnormal component may be transmitted to the repair service of the corresponding component (S860). When the turbidity sensor is determined to be abnormal (S855), sensor abnormality information may be transmitted to the repair service of the turbidity sensor (S860).

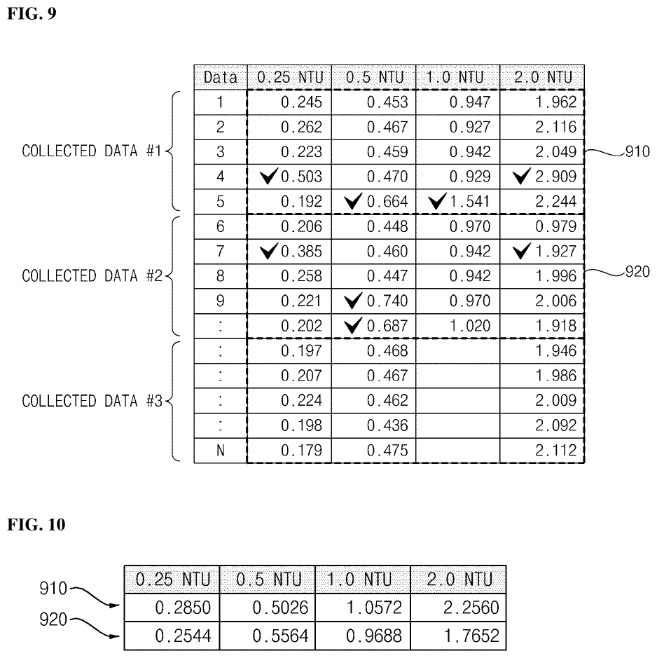

FIGS. 9 to 12 are diagrams for reference in describing an operation method of a water dispensing device according to an embodiment of the present disclosure.

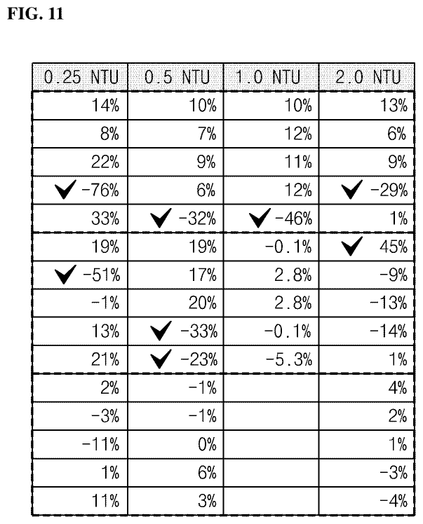

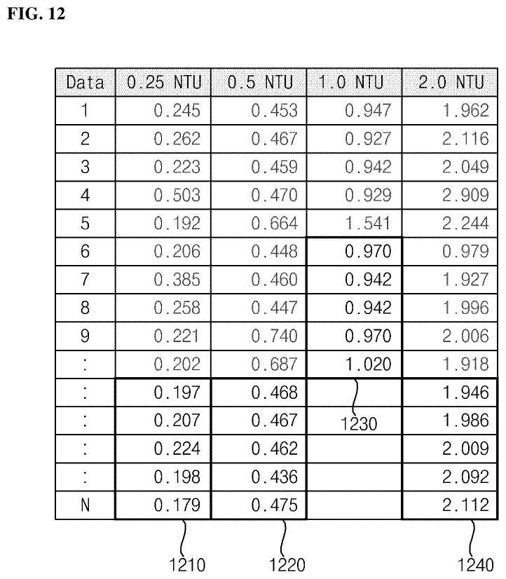

FIG. 9 illustrates data collected by introducing test water of each of 0.25 NTU, 0.5 NTU, 1.0 NTU, and 2.0 NTU into the turbidity sensor in an environment with external noise factors such as vibration, and FIG. 10 illustrates the average value of data 910 collected in the first sensing period of 0.25 NTU, 0.5 NTU, 1.0 NTU, and 2.0 NTU as illustrated in FIG. 9 and the average value of data 920 collected in the second sensing period. FIG. 11 shows a comparison value of the error ratio between the average value of FIG. 10 and the collected data of FIG. 9. FIG. 12 shows the final output sensing result data values 1210, 1220, 1230, 1240.

The sensing period may be set to the time during which the turbidity sensor may acquire a plurality of pieces of data. FIG. 9 illustrates a case where five pieces of data are acquired during one sensing time.

When there is no compensation and/or processing for data acquired by the turbidity sensor that is considered to be an error value, turbidity sensing may be determined inaccurately. Accordingly, the turbidity sensor may re-collect data until a data set without error values is acquired.

Referring to FIGS. 9 to 11, in the case of 0.25 NTU, 0.503 of the collected data #1 of the first sensing period is an error value that differs by 20% or more from the average value of 0.2850. Because the error value exists, the turbidity sensor re-collects data during the second sensing period.

In the collected data #2 during the second sensing period, 0.385 differs by 20% or more from the average value of 0.2544, so the turbidity sensor re-collects data during the third sensing period #3.

Referring to FIG. 12, in the case of 0.25 NTU, there is no error value in the data collected during the third sensing period #3 where the error ratio differs by 20% or more from the average value. Accordingly, the turbidity sensor may output sensing results based on the data collected in the third sensing period #3. Referring to FIG. 12, the turbidity sensor may output the average value of data 1210 collected in the third sensing period #3 as a sensing result.

Referring to FIGS. 9 to 11, in the case of 0.5 NTU, 0.664 of the collected data #1 of the first sensing period is an error value that differs by 20% or more from the average value of 0.5026. Because the error value exists, the turbidity sensor re-collects data during the second sensing period.

In the collected data #2 during the second sensing period, 0.740 and 0.687 differ by 20% or more from the average value of 0.5564, so the turbidity sensor re-collects data during the third sensing period #3.

Referring to FIG. 12, in the case of 0.5 NTU, there is no error value in the data collected during the third sensing period #3 where the error ratio differs by 20% or more from the average value. Accordingly, the turbidity sensor may output sensing results based on the data collected in the third sensing period #3. Referring to FIG. 12, the turbidity sensor may output the average value of data 1220 collected in the third sensing period #3 as a sensing result.

Referring to FIGS. 9 to 11, in the case of 1.0 NTU, 1.541 of the collected data #1 of the first sensing period is an error value that differs by 20% or more from the average value of 1.0572. Because the error value exists, the turbidity sensor re-collects data during the second sensing period.

Also in the collected data #2 during the second sensing period, there is no error value that differs by 20% or more from the average value of 0.9688. Accordingly, the turbidity sensor may output sensing results based on the data collected in the second sensing period #2. Referring to FIG. 12, the turbidity sensor may output the average value of data 1230 collected in the second sensing period #2 as a sensing result.

Referring to FIGS. 9 to 11, in the case of 2.0 NTU, 2.909 of the collected data #1 of the first sensing period is an error value that differs by 20% or more from the average value of 2.2560. Because the error value exists, the turbidity sensor re-collects data during the second sensing period.

In the collected data #2 during the second sensing period, 0.979 differs by 20% or more from the average value of 1.7652, so the turbidity sensor re-collects data during the third sensing period #3.

Referring to FIG. 12, in the case of 2.0 NTU, there is no error value in the data collected during the third sensing period #3 where the error ratio differs by 20% or more from the average value. Accordingly, the turbidity sensor may output sensing results based on the data collected in the third sensing period #3. Referring to FIG. 12, the turbidity sensor may output the average value of data 1240 collected in the third sensing period #3 as a sensing result.

According to an embodiment of the present disclosure, the turbidity sensor may remove error values and output accurate sensing results, and the control unit 60 may appropriately control the water dispensing device based on the sensing results of the turbidity sensor.

Hereinbefore, although preferred embodiments of the present disclosure have been illustrated and described, the present disclosure is not limited to the specific embodiments described above, and it goes without saying that persons having ordinary skills in the technical field to which the present disclosure pertains may implement the present disclosure by various modifications thereof without departing from gist of the present disclosure defined by the claims, and such modifications are not to be construed individually from the technical spirit and scope of the present disclosure.

1. A water dispensing device, comprising:
a water supply channel through which raw water supplied from a water supply source flows;
a filter which generates purified water by filtering the raw water supplied through the water supply channel;
a purified water channel through which the purified water passed through the filter flows; and
a water quality measuring unit which is connected to the purified water channel to measure a water quality of the purified water,
wherein the water quality measuring unit comprises a turbidity sensor, and the turbidity sensor collects N pieces of turbidity data during a first sensing time, outputs a sensing result on the basis of the N pieces of turbidity data when there is no abnormality in the collected N pieces of turbidity data, and re-collects the turbidity data to determine whether there is an abnormality when there is an abnormality in the collected N pieces of turbidity data.

2. The water dispensing device of claim 1, wherein the re-collection of the turbidity data is performed during a second sensing time that is longer than the first sensing time.

3. The water dispensing device of claim 1, wherein the turbidity sensor: calculates an average value of the N pieces of turbidity data; calculates an error ratio between the N pieces of turbidity data and the average value; and determines whether there is an abnormality when an absolute value of the calculated error ratio has a value exceeding a reference value.

4. The water dispensing device of claim 1, further comprising a control unit that stops operation of at least one of a compressor or a pump when there is an abnormality as a result of determining the abnormality after re-collecting the turbidity data.

5. The water dispensing device of claim 4, wherein, after the turbidity data is re-collected, when there is no abnormality as a result of determining the abnormality, a sensing result based on the re-collected turbidity data is output.

6. The water dispensing device of claim 4, wherein, when no normal data is collected until a third sensing period that is longer than the first sensing period, the control unit stops operation of at least one of the compressor or the pump.

7. The water dispensing device of claim 4, wherein the turbidity sensor collects the turbidity data in a state where at least one of the compressor or the pump is stopped from operating.

8. The water dispensing device of claim 7, wherein the control unit determines that there is an abnormality with the turbidity sensor when there is an abnormality in data collected in a state where at least one of the compressor or pump is stopped from operating, and determines that there is an abnormality with at least one of the compressor or the pump when there is no abnormality in the data collected in the state where at least one of the compressor or the pump is stopped from operating.

9. The water dispensing device of claim 8, wherein the control unit automatically connects a service of a component determined as an abnormality.

10. The water dispensing device of claim 1, wherein the sensing result is an average value of the N pieces of turbidity data.

11. The water dispensing device of claim 1, further comprising a sensing channel diverged from the water supply channel and through which the raw water flows, wherein the water quality measuring unit measures the water quality of the purified water when the purified water enters through the purified water channel and measures the water quality of the raw water when the raw water enters through the sensing channel.

12. The water dispensing device of claim 11, wherein the water quality measuring unit measures the water quality of the purified water after performing a rinse operation in which the purified water passes through at least once when measuring the water quality of the raw water.

13. The water dispensing device of claim 11, further comprising:
a switching valve for supplying the raw water to the water supply channel or the sterilization channel; and
a sensing valve for opening and closing the sensing channel.

14. The water dispensing device of claim 1, further comprising:
a water outlet through which the purified water is discharged;
a water outlet channel guiding the purified water to the water outlet; and
a drainage channel diverged from the water outlet channel between the water quality measuring unit and the water outlet through which the raw water or the purified water is drained.

15. The water dispensing device of claim 14, further comprising a water outlet valve selectively supplying the raw water or the purified water to the water outlet channel and the drainage channel.

16. The water dispensing device of claim 14, further comprising:
a hot water channel diverged from the purified water channel at one side;
a hot water module provided in the hot water channel to heat purified water passing through the hot water channel;
a cold water channel diverged from the purified water channel at one side; and
a cold water module provided in the cold water channel to cool purified water passing through the cold water channel.

17. The water dispensing device of claim 14, further comprising a drainage pump disposed in the drainage channel.

18. The water dispensing device of claim 1, wherein the turbidity sensor irradiates light to the raw water or a portion of the purified water and senses turbidity based on a pattern of scattered light received.
